(19) United States
(12) Patent Application Publication
(10) Pub. No.: US 2003/0158297 A1
(43) Pub. Date: Aug. 21, 2003

(54) Process for producing fine powdery functional material

(75) Inventors: Masataka Sano, Hamamatsu-shi (JP); Hiroki Miyamatsu, Hamamatsu-shi (JP); Kimi Yoshida, Hamamatsu-shi (JP); Nobuo Goto, Hamamatsu-shi (JP)

Correspondence Address: ARMSTRONG,WESTERMAN & HATTORI, LLP, 1725 K STREET, NW, SUITE 1000, WASHINGTON, DC, 20006, US

(73) Assignee: KABUSHIKI KAISHA ERUBU, Hamamatsu-shi (JP)

(21) Appl. No.: 10/368,383

(22) Filed: Feb. 20, 2003

(30) Foreign Application Priority Data
Feb. 20, 2002 (JP) 2002-043530

(51) Int. Cl. A61K 6/00; C08K 3/34

(52) U.S. Cl. 523/340; 524/492; 424/401; 424/405

(57) ABSTRACT

An industrially advantageous process for producing at once a composite material of an organic component having functionality and a ceramic component based on an idea different from the conventional techniques, in which (C) an aqueous slurry in a fine droplet form containing (A) an organic component having a functionality and (B) a ceramic component, is supplied into a chamber of a spray dryer, and simultaneously hot air is blown into the chamber, whereby drying and fine powderization of the droplets are attained through contact of the droplets with the hot air in the chamber, to produce at once a fine powdery hybrid material through hybridization of the organic component (A) and the ceramic component (B).

### BACKGROUND OF THE INVENTION

1. Field of the Invention

The present invention relates to an industrial process for producing at once a fine powdery hybrid material containing an organic component having a functionality, such as deodorant activity and antimicrobial activity, and a ceramic component.

2. Description of Conventional Art

Organic components represented by catechin and saponin have such functionalities as deodorant activity and antimicrobial activity, and a material having a functionality, such as deodorant activity and antimicrobial activity, can be produced in such a manner that they are internally added upon molding a molded product of, for example, fibers, and they are attached to various kinds of objective products along with a suitable binder.

In this case, there is such a problem that the functionality lacks persistence by losing the organic component upon using under contact with water. the material having the organic component internally added or attached.

The inventors have made various proposals for attaining the persistence in such a manner that the organic component, such as catechin and saponin, is formed into a composite material with a ceramic component, and the composite material is internally added upon molding a molded product or is attached to various kinds of objective products along with a suitable binder.

For example, JP-A-2000-204277 discloses such a functional molded article that is formed with a melt molded article of a molding resin containing a functional component having deodorant activity or antimicrobial activity selected from a catechin compound, a saponin compound, tea leaf powder, a tea leaf extract and tannin (tannic acid), and a ceramic component, JP-A-2000-271201 discloses such a functional molded article that contains a water swelling clay mineral having carried thereon a functional component having antimicrobial activity, bioactivity or deodorant activity selected from a catechin compound, a saponin compound, tea leaf powder, a tea leaf extract and tannin (tannic acid), and JP-A-2001-159029 discloses composite filaments.

In the case where an organic component, such as catechin and saponin, is formed into a composite material with a ceramic component, a product composite material is produced through many process steps, i.e., the organic component is formed into a mixture with a ceramic component (such as ceramic particles, an inorganic sintering assistant and an inorganic aggregating agent) and is preferably aggregated, and then subjected to a heat treatment at a high temperature by using a crucible until dehydration, and the product is pulverized by mechanical means and then finally classified.

However, such process steps bring about a high load in equipments, labor (man-hour) and time to increase the cost of the product, but the necessary steps cannot be omitted as long as ceramics are handled. Therefore, there is a limitation in rationalization of the production process.

### SUMMARY OF THE INVENTION

Under the circumstances, an object of the present invention is to provide an industrially advantageous process for producing at once a composite material of an organic component having a functionality and a ceramic component based on an idea different from the conventional techniques.

The present invention relates to a process for producing fine powdery functional material containing a step of supplying (C) an aqueous slurry in a fine droplet form containing (A) an organic component having a functionality and (B) a ceramic component into a chamber of a spray dryer, and simultaneously blowing hot air into the chamber, to attain drying and fine powderization of the droplets through contact with the hot air in the chamber, to form at once a fine powdery hybrid material through hybridization of the organic component (A) and the ceramic component (B).

### BRIEF DESCRIPTION OF THE DRAWINGS

FIG. 1 is an explanatory diagram showing an example of a spray dryer used in the process of the present invention.

FIG. 2 is an enlarged explanatory diagram showing a four-fluid nozzle used in the spray dryer shown in FIG. 1.

### DESCRIPTION OF THE PREFERRED EMBODIMENTS

The present invention will be described in detail below.

Organic Component (A)

Examples of the organic component (A) having a functionality include functional components derived from plants, animals and microorganisms having at least one capability of deodorant activity (including deodorizing activity, offensive odor removing activity and toxic gas removing activity), antimicrobial activity (including antibacterial activity, sterilization activity, bacteriostatic activity, antifungal activity and antivirus activity), relaxation activity, aromatherapy activity, antiallergic activity, antioxidation activity, antiinflammationactivity, moisturizingactivityand noxious small animal repelling activity.

The organic component (A) can be obtained by extracting an objective effective component from plants, animals and microorganisms by steam distillation, solvent extraction, squeezing and other methods. Synthetic materials equivalent to the functional components contained in plants and animals can also be used as the organic component (A).

Specific examples of the organic component (A) include:

an effective component derived mainly from tea, such as a catechin compound, a saponin compound, tea leaf powder, a tea leaf extract, tannin (tannic acid) and caffeine;

an essential oil and an extract derived from tea tree, pine, clove, sage, nutmeg, ginkgo leaves, rice hull, cibol, Alpina galanga, coffee lime, coffee beans, guava tea, checker tree, stoneweed roots, bamboo and bamboo leaves;

a glycoside derived from Western mustard;

hyaluronic acid;

a polysaccharide derived from agaricus;

a protein derived from plants, animals or microorganisms and a decomposed product thereof, such as a milt protein (protamine), a decomposed product of pectin, albumen, lysozyme, which is a low molecular weight basic protein of sycamore and is an enzyme, and a polyamino acid (such as polylysine and polyglutamic acid) or a salt thereof;

bovine bezoar;

an amino acid or a derivative thereof, such as a metallic soap (the metallic ion is preferably an antibacterial metal, such as Ag), e.g., an N-stearyl-L-glutamic acid salt, an N-lauryl-L-glutamic acid salt and an N-acylmethyltaurine derivative, a γ-aminobutyric acid (such as one derived from brown rice), and teanin(γ-glutamylethylamide);

kojic acid (containing 5-oxy-2-oxymethyl-γ-pyrone as a main component) and a decomposition product of red koji;

L-ascorbic acid (vitamin C), a water soluble derivative thereof (such as Na, K, Mg, Ca and Al salts of L-ascorbic acid phosphate ester, Na, K, Mg, Ca and Al salts of L-ascorbic acid sulfate ester, and Na, K, Mg, Ca and Al salts of L-ascorbic acid), and vitamin D;

an essential oil and an extract derived from plants, such as anise, amyris, angelica, benjamin tree, immortelle, ylang-ylang, elemi, origanum, orange, camomile, cajuput, garlic, cardamom, galbanum, camphor (camphor tree), caraway, carrot seed, guaiacwood, cumin, clary sage, grapefruit, coriander, cypress, sandalwood (sandal tree), santolina, cedarwood, citronella, cinnamon, jasmine, juniper, ginger, star anise, spike lavender, spearmint, geranium, thyme, tagetes, tarragon, tangerine, dill, terebinth, niaouli, lentiscus, neroli, violet, basil, birch, patchouli, verbena, rose, palmarosa, hyssop, pimento, fir (fir tree), foeniculum (fennel), petitgrain, blackpepper, vetivert, peppermint, bergamot, limeblossom, marjoram, myrtle, mandarin, melissa, myrrh, yarrow, eucalyptus, lime, raspberry, lavandine, lavender, litseacubeba, lemon, lemongrass, rosewood, rosemary and laurel;

an essential oil and an extract derived from plants, such as Japanese laurel, obeche, madder, Mallotus japonicus, Rehmannia glutinosa, aloe, apricot, epimedium, Japanese knotweed, yew tree, sycamore, Achyranthes fauriei, curcuma, satsuma orange, sicklepod, sophora, coptis, plantain, Atractylodes japonica, Panax ginseng, Saint John's wort, persimmon, Uncaria rhynchophylla, ground ivy, valerian, leaf mustard, Pinellia ternata, Artemisia capillaris, licorice, Trichosanthes kirilowii, Japanese bellflower, catalpa, sorrel, Amur cork tree, Astragalus membranaceus, agrimony, boxthorn, kudzu vine, gardenia, spicebush, mulberry tree, cranesbill, Scutellaria baicalensis, Evodia rutaecarpa, thrumwort, Zizyphus jujuba, smilax, Curnus officinalis, zanthoxylum, Japanese basil, peony root, Ophiopogon japonicus, ginger, honeysuckle, field horsetail, Japanese parsley, Cnidium officiale, sialid, pieplant, bigarade, devil's-walking-stick, Sulvia miltiorrhiza, dandelion, cupseed, Polygonum multiflorum, Japanese silverleaf, Angelica sinensis, dokudami, aconite, jujube, Polygonatum falcatum, nandina, bitterwood, elder tree, garlic, Japanese rose, tear grass, liliaceae, Alpinia japonica, swordweed, nut grass, Glehnia littoralis, Isodom japonicus, bellbind, Japanese medlar, betel, boneset, safflower, peony, mahuang, silver vine, Bupleurum, althea, Leonurus sibiricus, peach, yam, meadow saxifrage, mugwort, bitterwort, Sophora flavescens, Bupleurum scorzoneraefolium, phellodendron bark, mint, Rehmannia glutinosa, curcuma, Malva verticillata, tuckahoe, cassia, catear, sappanwood, forsythia, Anemarrhena asphodeloids, clove, white nandina, catechu, bamboo leaves, Astragalus membranaceus, angelica dahurica root, Schizonepeta tenuifolia, Dictamnus dasycarpus, pepper, Japanese horseradish, celery, parsley, oregano, citrus seeds, knotweed, soybean, grape pericarp, seeds of Psoralea corylifolia, Phyllostachys pubescens, rice hull, bayberry, storax, Artemisia capillaris, Japanese cypress, magnolia, forsythia, apple, litchi and monkeyhead mushroom;

a traditional prescription of extracts of herbal medicines and Chinese medicines, such as Ohrengedoku-toh, Shofu-san, Makyohyokukan-toh, Kakkon-toh, Jumihaidoku-toh, Keigairengyo-toh, Kujin-toh, Keibouhaidoku-toh and Jashoushi-toh;

a synthetic compound equivalent to a functional component contained in plants and animals, such as allyl isothiocyanate (effective component of Japanese horseradish and mustard), shikonin, hinokitiol and various kinds of perfumes;

a polysaccharide, such as chitin and chitosan;

propolis, which is a glue substance formed by mixing a vegetable resin component collected by honey bees and their secretion product; and

a plant pulverized product, an animal pulverized product and a microorganism or a pulverized product thereof, such as a rice hull pulverized product, a bamboo pulverized product, bamboo charcoal pulverized product, a pulverized product of baked pilgrim shells, a litchi pulverized product and an agaricus pulverized product.

Ceramic Component (B)

Examples of the ceramic component (B) include the following, which are used solely or in combination of two or more of them.

ceramics fine particles capable of being formed into a colloidal or sol aqueous dispersion, for example, an inorganic agglomerate in a sol form or a solution form, such as colloidal silica and alumina sol, preferably colloidal silica, an inorganic agglomerate in a solution form and a silicate (such as sodium silicate, potassium silicate and lithium silicate) in a solution form;

silica gel obtained through water-containing silicic acid;

an inorganic sintering assistant, for example, a polyvalent metallic salt (such as aluminum, zinc, magnesium, calcium and manganese) and an alkali metal salt of an inorganic salt, such as phosphoric acid, sulfuric acid, nitric acid and carbonic acid, a fluoride and a silicofluoride of an alkali metal, such as lithium, sodium and potassium, and an alkaline earth metal, such as magnesium and calcium;

a clay mineral (such as kaolin and bentonite), an oxide (such as alumina, titania, silica, zirconia, magnesia and zinc oxide), a hydroxide (such as hydroxides of aluminum, zinc, magnesium, calcium and manganese), a composite oxide (such as alum), a nitride (such as silicon nitride and boron nitride), a carbide (such as silicon carbide and boron carbide), a silicide, a boride, zeolite, cristobalite, diatom earth and a polyvalent metallic salt of silicic acid (such as an aluminum salt, a zinc salt, a magnesium salt, a calcium salt and a manganese salt);

a tablet mineral having a low hardness degree and cleaving property, such as talc and natural mica;

a fluoride of talc or synthetic mica having a fluorine atom;

a clay mineral having such a nature that it is swollen through absorption of water, such as sepiolite, vermiculite, bentonite and sericite;

zinc oxide;

piezoelectric ceramics;

far infrared ray radiation ceramics; and

slightly radioactive ceramics.

A representative example of the ceramic component (B) is colloidal silica, and an embodiment using colloidal silica as at least a part of the ceramic component (B) is important in the present invention. In this case, when a mixture of colloidal silica and other ceramics is used, there are some cases where, upon drying the droplets, the objective material is formed into further fine powder, the size of which is smaller than the size defined by the size of the droplets. Therefore, the procedure can be utilized for controlling the particle diameter of the fine powder.

Process for Producing Fine Powdery Functional Material

In the process for producing fine powdery functional material according to the present invention, (C) an aqueous slurry containing (A) an organic component having a functionality and (B) a ceramic component, in a fine droplet form is supplied into a chamber of a spray dryer, and simultaneously hot air is blown into the chamber, whereby drying and fine powderization of the droplets are attained through contact of the droplets with the hot air in the chamber. Consequently, a fine powdery hybrid material through hybridization of the organic component (A) and the ceramic component (B) is formed at once.

Examples of the spray dryer include not only a vertical falling type, in which the droplets vertically fall down, but also other various types, such as a blowing up type, a horizontal type and a cyclone type.

The diameter of the droplets thus made fine is generally classified into spray when it exceeds 10 μm and mist when it is 10 μm or less, and the droplets are particularly preferably in the form of the mist.

The formation of the droplets is generally carried out by using a rotating disk (change in rotation number), a pressure nozzle (pressure of liquid), a two-fluid nozzle (gas pressure), and a four-fluid nozzle (gas pressure).

Among these, a four-fluid nozzle, which is one of nozzles utilizing compressed air, is particularly preferably used because it can spray a large amount of droplets in the form of mist. The four-fluid nozzle, which will be described below, is described in Kagaku Souchi (Chemical Equipments), June of 2000, p. 60 to 65, by authors of Fujisaki Electric Co., Ltd., and also described in detail in technical brochures of the company.

In the four-fluid nozzle, two gas flow paths and two fluid flow paths, i.e., four flow paths in total are symmetrically provided in a nozzle edge having a knife form, in which a collision point is constituted at a tip end of the edge, at which bevels as a flowing surface of the gas and the liquid and the respective fluids are converged. The nozzle edge is preferably provided with a linear portion having a suitable length (for example, from 1 to 200 mm in edge length) depending on the spraying amount. The high-speed gas fluid ejected from a gas slit thinly elongates the liquid, which upwells from a liquid slit, on a fluid flowing surface along mixing. The thinly elongated liquid is further made fine with a shock wave generated at the collision point at the tip end of the edge and then borne away as droplets having a diameter of several micrometers.

The use of a four-fluid nozzle is particularly advantageous because of the following points: a minute droplet diameter of several micrometers in terms of an average particle diameter can be obtained; droplets having a sharp particle size distribution can be obtained; the droplet diameter can be arbitrarily controlled by the gas/liquid ratio; a large amount of droplets can be sprayed with one nozzle; droplets can be continuously sprayed for a long period of time without clogging with attachments owing to the self-washing and external mixing system; plural kinds of liquids can be sprayed with mixing at the tip end of the nozzle; the spraying pattern can be changed by changing the two air amounts; and a nozzle with a different spraying amount can be produced with maintenance of the performance. The edge nozzle may be either a straight type or a circle type.

The ratio (in terms of solid content) of the organic component (A) and the ceramic component (B) is, while it can be variously set, preferably 0.1 to 200 parts by weight, more preferably 1 to 180 parts by weight, and particularly preferably 2 to 160 parts by weight, of the organic component (A) per 100 parts by weight of the ceramic component (B). When the proportion of the organic component (A) is too small, the functionality becomes insufficient. When the proportion of the organic component (A) is too large, the proportion of the ceramic component (B) becomes relatively short to cause insufficient formation of a composite material, whereby the persistence becomes short.

A solvent for the aqueous slurry (C) is generally water, and an appropriate amount of an organic solvent (such as an alcohol) may be contained.

The solid content of the aqueous slurry (C) is, while it is not particularly limited, generally 1 to 70% by weight, preferably 3 to 60% by weight, and particularly preferably 5 to 50% by weight, from the standpoint of fine powderization and heat energy.

The temperature upon spraying is preferably set at 170 to 300° C. (more preferably 180 to 290° C., and particularly preferably 190 to 280° C.) as an inlet temperature of the hot air. The exhaust temperature is preferably set at 65 to 250° C. (more preferably 80 to 220° C., and particularly preferably 90 to 200° C.) and a temperature that is lower than the inlet temperature by 30° C. or more (preferably 50° C. or more). When the temperature of the hot air is too low, the drying rate becomes too low, and the formation of a composite material (i.e., hybridization) becomes insufficient, whereby the persistence becomes short upon using the material under contact with water. When the temperature of the hot air is too high, on the other hand, there is a possibility that the organic component (A) is degenerated or vaporized. The objective product can be efficiently obtained under the temperature conditions described in the foregoing.

The average particle diameter of the objective product derived from the chamber is preferably controlled to 20 μm or less, and more preferably 15 μm or less. It is also conveniently controlled to 10 μm or less. The particle diameter can be controlled by controlling the size of the droplets and the particle diameter of the ceramic component (B) in the aqueous slurry (C). The lower limit thereof is not particularly limited, and it can be easily about 1 μm or can also be in a submicron order.

The objective product (i.e., a fine powdery hybrid material) can be used in various forms, for example, the powdery material is used as it is; the powdery material is enclosed in a bag or sandwiched in layers; the powdery material is subjected to granulation or molding; the powdery material is internally added to (kneaded with) a polymer component to form a molded article; and a coating composition is formed with the powdery material with the use of a binder depending on necessity, and the coating composition is coated on impregnated in an arbitrary object.

### EXAMPLES

The invention will be described in more detail with reference to the following examples.

### Examples 1 to 3

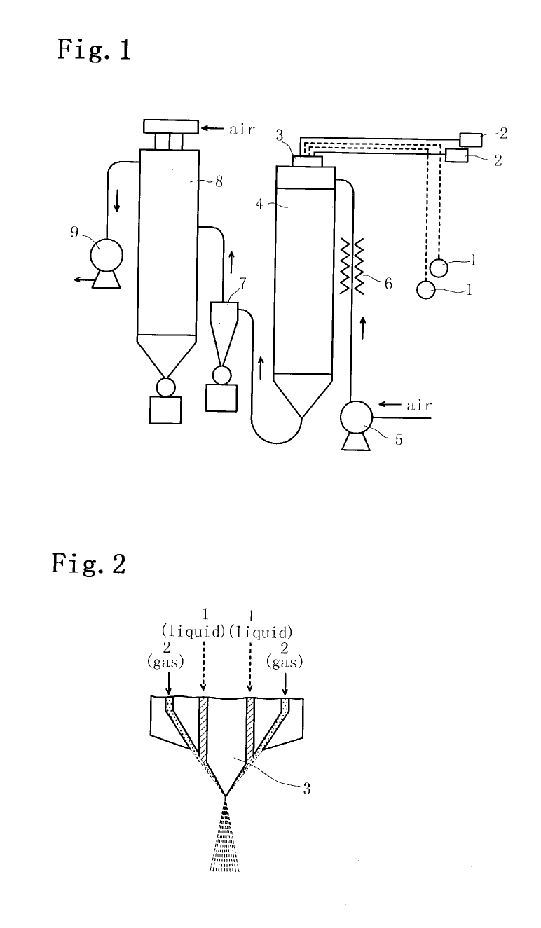

FIG. 1 is an explanatory diagram showing an example of a spray dryer used in the process of the present invention.

FIG. 2 is an enlarged explanatory diagram showing a four-fluid nozzle used in the spray dryer shown in FIG. 1.

In FIGS. 1 and 2, numeral 1 denotes a liquid supplying system, 2 denotes a gas supplying system, 3 denotes a nozzle (four-fluid nozzle), 4 denotes a main body of the spray dryer (chamber), 5 denotes an air blower, 6 denotes a heater, 7 denotes a cyclone, 8 denotes a bag filter, and 9 denotes an air discharger.

The liquid supplying system 1 and the gas supplying system 2 each contains two flow paths, and spraying is carried out at a tip end of the nozzle 3 as shown in FIG. 2. Recovery (collection) of the product can be attained by both or one of the cyclone 7 and the bag filter 8.

10 parts by weight of tea catechin having a purity of 30% by weight was added to 100 parts by weight of an aqueous colloid liquid of colloidal silica having a solid content of 40% by weight, and they were mixed with a homogenizer for 3 minutes to prepare an aqueous slurry.

The aqueous slurry was spray-dried by using a laboratory spray dryer equipped with a four-fluid nozzle, “Micromist Dryer MDL-050 Type M”, produced by Fujisaki Electric Co., Ltd., under the following conditions, and the objective product was recovered with the cyclone (CY) and/or the bag filter (BF).

| | | |
|---|---|---|
| Edge length: | 2 | mm |
| Air slit: | 0.2 | mm |
| Liquid slit: | 0.2 | mm |
| Air amount: | about 25 | L/min |
| Sprayed liquid amount: | about 10 | mL/min |
| Air pressure: | about 0.7 | MPa |
| Hot air amount: | about 1 | m³/min |

The conditions and results are shown in Table 1 below. The inlet temperature of the hot air was set at about 200° C. or about 250° C. because it had been experimentally confirmed that raw catechin remained in a large amount due to insufficient formation of a composite material (hybridization) at about 150° C. The recovered amount in Example 3 became 971.6 g (=94.6+877) after 2 hours, and 1,693.6 g (=94.6+877+722). The particle size distribution was measured by using “SALD-200V ER”, produced by Shimadzu Corp.

TABLE 1

| | | Example 1 | Example 2 | Example 3 | | |
|---|---|---|---|---|---|---|
| Time (min) | | 5 | 5 | 60 | 120 | 180 |
| Inlet temperature (° C.) | | 200 | 250 | 250 | 250 | 250 |
| Exhaust temperature (° C.) | | 85 | 107 | 99 | 102 | 111 |
| Tower pressure (kPa) | | 0.4 | 0.5 | 0.8 | 0.7 | 0.6 |
| Supplied air amount (m³/min) | | 1.1 | 0.85 | 0.85 | 0.85 | 0.87 |
| Air 1 | Pressure (MPa) | 0.69 | 0.69 | 0.69 | 0.69 | 0.68 |
| | Flow amount (NL/min) | 80 | 80 | 80 | 80 | 80 |
| Air 2 | Pressure (MPa) | 0.69 | 0.69 | 0.69 | 0.69 | 0.69 |
| | Flow amount (NL/min) | 80 | 80 | 80 | 80 | 80 |
| Liquid 1 | Flow amount (ml/min) | 20 | 20 | 20 | 20 | 10 |
| Liquid 2 | Flow amount (ml/min) | 20 | 20 | 20 | 20 | 10 |

| | | | | | |
|---|---|---|---|---|---|
| Internal pressure in BF (kPa) | 0.3 | 0.4 | 1 | 0.7 | 0.5 |
| Recovered amount (g) | 46.8 | 32.6 | 94.6 | 877 | 722 |
| Median diameter (μm) | 5.614 | 6.075 | 5.808 | 6.412 | 5.949 |
| Mode diameter (μm) | 6.512 | 6.514 | 6.512 | 7.527 | 6.512 |
| Average particle diameter (μm) | 5.598 | 6.298 | 5.873 | 6.624 | 6.217 |
| Standard deviation | 0.293 | 0.324 | 0.302 | 0.329 | 0.321 |
| 25% Diameter (μm) | 3.680 | 3.914 | 3.782 | 4.071 | 3.874 |
| 50% Diameter (μm) | 5.614 | 6.075 | 5.806 | 6.412 | 5.949 |
| 75% Diameter (μm) | 8.018 | 9.114 | 8.519 | 9.734 | 8.899 |

### Examples 4 and 5

40 parts by weight of an aqueous solution of a polyamino acid (polylysine) having a concentration of 25% by weight was added to and mixed with 100 parts by weight of an aqueous colloid liquid of colloidal silica having a solid content of 40% by weight to prepare an aqueous slurry. The aqueous slurry was spray-dried by using a laboratory spray dryer, “Micromist Dryer MDL-050 Type B”, produced by Fujisaki Electric Co., Ltd., in the same manner as in Example 1. The conditions and the results are shown in Table 2 below.

TABLE 2

| | | | Example 4 | Example 5 |
|---|---|---|---|---|
| Time (min) | | | 9 | 4 |
| Inlet temperature (° C.) | | | 201 | 201 |
| Exhaust temperature (° C.) | | | 90 | 85 |
| Tower pressure (kPa) | | | 5.0 | 3.8 |
| Supplied air amount (m³/min) | | | 0.75 | 0.80 |
| | Air 1 | Pressure (MPa) | 0.67 | 0.67 |
| | | Flow amount (NL/min) | 80 | 80 |
| | Air 2 | Pressure (MPa) | 0.67 | 0.67 |
| | | Flow amount (NL/min) | 80 | 80 |
| | Liquid 1 | Flow amount (ml/min) | 20 | 20 |
| | Liquid 2 | Flow amount (ml/min) | 20 | 20 |

| | | |
|---|---|---|
| Recovered amount (g) | 151.9 | 262.3 |
| Median diameter (μm) | 19.662 | 13.682 |
| Mode diameter (μm) | 27.728 | 23.988 |
| Average particle diameter (μm) | 15.873 | 12.468 |
| Standard deviation | 0.395 | 0.414 |
| 25% Diameter (μm) | 8.474 | 6.895 |
| 50% Diameter (μm) | 19.662 | 13.682 |
| 75% Diameter (μm) | 31.811 | 26.111 |

### Examples 6 to 10

Spray drying was carried out in the same manner as in Example 1 except that the following compositions were used as the aqueous slurry. The condition and the results are shown in Table 3.

Example 6: 17.5% by weight of tea catechin having a purity of 30% by weight, 10% by weight of an aqueous colloid liquid of colloidal silica having a solid content of 40% by weight, 12.5% by weight of pulverized talc having an average particle diameter of 0.5 μm, and 60% by weight of water

Example 7: 17.5% by weight of tea catechin having a purity of 30% by weight, 5% by weight of an aqueous colloid liquid of colloidal silica having a solid content of 40% by weight, 12.5% by weight of pulverized talc having an average particle diameter of 0.5 μm, and 65% by weight of water

Example 8: 17.5% by weight of tea catechin having a purity of 30% by weight, 12.5% by weight of pulverized silica having an average particle diameter of 1.5 μm, 5% by weight of an aqueous colloid liquid of colloidal silica having a solid content of 40% by weight, and 65% by weight of water

Example 9: 25% by weight of polylysine and 75% by weight of an aqueous colloid liquid of colloidal silica having a solid content of 40% by weight

Example 10: 25% by weight of polylysine, 5% by weight of pulverized talc having an average particle diameter of 0.5 μm, and 70% by weight of an aqueous colloid liquid of colloidal silica having a solid content of 40% by weight

TABLE 3

| | Example 6 | Example 7 | Example 8 | Example 9 | Example 10 |
|---|---|---|---|---|---|
| Inlet temperature (° C.) | 225 | 225 | 224 | 221 | 221 |
| Exhaust temperature (° C.) | 106 | 102 | 100 | 104 | 108 |
| Median diameter (μm) | 4.146 | 5.765 | 1.596 | 12.371 | 17.008 |
| Mode diameter (μm) | 4.216 | 5.633 | 1.529 | 8.700 | 8.700 |
| Average particle diameter (μm) | 4.133 | 6.141 | 1.608 | 13.401 | 17.610 |
| Standard deviation | 0.275 | 0.363 | 0.221 | 0.353 | 0.474 |
| 25% Diameter (μm) | 2.778 | 3.570 | 1.093 | 7.702 | 7.987 |
| 50% Diameter (μm) | 4.146 | 5.765 | 1.596 | 12.371 | 17.008 |
| 75% Diameter (μm) | 5.978 | 9.212 | 2.350 | 25.809 | 37.796 |

In the case using only 100% by weight of an aqueous colloid liquid of colloidal silica having a solid content of 40% by weight (without the organic component (A)), under conditions of an inlet temperature of 250° C. and an exhaust temperature of 105° C., the median diameter was 7.490 μm, the mode diameter was 7.527 μm, the average particle diameter was 8.589 μm, the standard deviation was 0.243, the 25% diameter was 5.976 μm, the 50% diameter was 7.490 μm, and the 75% diameter was 9.851 μm.

### EFFECTS OF THE INVENTION

According to the present invention, a fine powdery hybrid material through hybridization of the organic component (A) having a functionality and the ceramic component (B) can be produced at once to provide considerable advantages in productivity, man-hour, equipments, heat energy and process time, in comparison to the conventional process requiring mixing, heat treatment, pulverization and classification steps. In particular, it is advantageous when a four-fluid nozzle is used as a nozzle.

In the resulting fine powdery hybrid material, the organic component (A) is protected by hybridization with the ceramic component (B), and thus the material exerts excellent persistence upon using the material under contact with water.

What is claimed is:

1. A process for producing fine powdery functional material comprising a step of supplying (C) an aqueous slurry in a fine droplet form comprising (A) an organic component having a functionality and (B) a ceramic component into a chamber of a spray dryer, and simultaneously blowing hot air into the chamber, to attain drying and fine powderization of the droplets through contact with the hot air in the chamber, so as to form at once a fine powdery hybrid material through hybridization of the organic component (A) and the ceramic component (B).

2. A process for producing fine powdery functional material as claimed in claim 1, wherein a nozzle for forming the droplets is a four-fluid nozzle having two gas flow paths and two fluid flow paths.

3. A process for producing fine powdery functional material as claimed in claim 1, wherein an inlet temperature of the hot air is set at 170 to 300° C., and an exhaust temperature is set at 65 to 250° C. and lower than the inlet temperature by 30° C. or more.

4. A process for producing fine powdery functional material as claimed in claim 1, wherein an average particle diameter of an objective product derived from the chamber is controlled to 20 μm or less.

5. A process for producing fine powdery functional material as claimed in claim 1, wherein the organic component (A) is a component having a functionality derived from plants, animals and microorganisms, or a synthetic material equivalent to a functional component contained in plants and animals.

6. A process for producing fine powdery functional material as claimed in claim 1, wherein at least a part of the ceramic component (B) is ceramics fine particles capable of being formed into a colloidal or sol aqueous dispersion.

7. A process for producing fine powdery functional material as claimed in claim 6, wherein at least a part of the ceramic component (B) is colloidal silica.

8. A process for producing fine powdery functional material as claimed in claim 6, wherein the ceramic component (B) is a mixture of colloidal silica and other ceramics.
